(19) United States
(12) Patent Application Publication
(10) Pub. No.: US 2023/0364391 A1
(43) Pub. Date: Nov. 16, 2023

(54) Dressing Optimized to Facilitate Line Draw Through an Integrated Catheter with an Angled Extension

(71) Applicant: Becton, Dickinson and Company, Franklin Lakes, NJ (US)

(72) Inventors: Dorien Gemma Butter, Millcreek, UT (US); Erica E. Neumann, Sandy, UT (US); Benjamin P. Hopwood, West Valley City, UT (US); John M. Lackey, West Valley City, UT (US); Ralph L. Sonderegger, Farmington, UT (US); Jonathan Karl Burkholz, Salt Lake City, UT (US)

(21) Appl. No.: 18/196,676

(22) Filed: May 12, 2023

Related U.S. Application Data
(60) Provisional application No. 63/342,343, filed on May 16, 2022.

(51) Int. Cl. A61M 25/02

(52) U.S. Cl. CPC A61M 25/02; A61M 2025/0246; A61M 2025/0266

(57) ABSTRACT

Provided herein is a dressing for use with a catheter assembly, including a base defining a perimeter and an interior, the base comprising a bottom surface configured to contact skin of a patient and comprising a first opening in the perimeter, and a cover portion attached to the base and configured to cover a site of a catheter insertion, the cover portion comprising a second opening, the second opening configured to align with the first opening in the base when the dressing is applied to a patient’s skin.

### CROSS-REFERENCE TO RELATED APPLICATION

The present application claims priority to United States Provisional Application Serial No. 63/342,343, entitled “Dressing Optimized to Facilitate Line Draw Through an Integrated Catheter with an Angled Extension”, filed May 16, 2022, the entire disclosure of which is hereby incorporated by reference in its entirety.

### BACKGROUND OF THE INVENTION

### Field of the Invention

Provided herein are devices and systems for use in vascular access, and, in particular, dressings and vascular access systems including such dressings.

### Description of Related Art

Properly dressing a catheter is important for a catheter’s dwell time, minimizing the risk for patient discomfort, dislodgment, and phlebitis among other conditions. Dressings can be designed for integrated catheters with a 45-degree side port. The angled slit in these dressings allows for them to be applied around the extension set, while maintaining a seal that helps prevent microbial ingress. However, the slit often sticks to the extension set during application, resulting in added manipulation of the catheter before the dressing is properly secured. This increases the risk for dislodgment and skin irritation. In addition, existing dressings intend alignment of a nonwoven border with the wings of the adapter and alignment of the slit with the extension set. This alignment is not intuitive for many users, often resulting in improper use.

Further, for many typical dressings, visibility is limited to the area around the insertion site. Having visibility around the extension set and nose of the adapter is important to monitor the health of a patient’s catheter insertion site. Visibility along the path of the line draw device allows for tracking and troubleshooting if any problems arise. Accordingly, there is a need in the field for dressings that allow for improved visualization and securement of an integrated catheter with an extension set.

### SUMMARY OF THE INVENTION

Provided herein is a dressing for use with a catheter assembly, including a base defining a perimeter and an interior, the base comprising a bottom surface configured to contact skin of a patient and comprising a first opening in the perimeter, and a cover portion attached to the base and configured to cover a site of a catheter insertion, the cover portion comprising a second opening, the second opening configured to align with the first opening in the base when the dressing is applied to a patient’s skin.

In accordance with an embodiment of the present invention, the second opening is configured to form a seal around a portion of a catheter assembly that passes through the first opening.

In accordance with an embodiment of the present invention, the perimeter defines a shape that is not a square, and wherein the portions of the base defining the first opening are arranged such that the portions form an oblique angle.

In accordance with an embodiment of the present invention, the base and/or the cover portion comprise an adhesive on a bottom surface thereof.

In accordance with an embodiment of the present invention, the base and/or the cover portion further comprise a removable backing at least partially covering the adhesive.

In accordance with an embodiment of the present invention, the removable backing includes at least two sections, and wherein the first opening and/or the second opening at least partially separate the at least two sections.

In accordance with an embodiment of the present invention, the base includes a non-woven material.

In accordance with an embodiment of the present invention, the base defines an interior portion substantially having a shape of a catheter adapter.

In accordance with an embodiment of the present invention, the base and/or the cover portion further includes one or more alignment markers.

In accordance with an embodiment of the present invention, the base includes two alignment markers arranged on opposite sides of the interior.

Also provided herein is a kit including at least one package containing a catheter assembly including a catheter adapter which includes a distal end, a proximal end, a lumen arranged between and in fluid communication with the distal end and the proximal end, a port arranged between the proximal end and the distal end at an angle relative to the lumen, a conduit in fluid communication with the port, and a catheter arranged at the distal end of the catheter adapter and in fluid communication with the lumen. The kit further includes a dressing having a base defining a perimeter and an interior, the base comprising a bottom surface configured to contact skin of a patient and comprising a first opening in the perimeter, and a cover portion attached to the base and configured to cover a site of a catheter insertion, the cover portion comprising a second opening, the second opening configured to align with the first opening in the base when the dressing is applied to the patient’s skin.

In accordance with an embodiment of the present invention, the dressing is configured to form a seal around a portion of a catheter assembly that passes through the first opening.

In accordance with an embodiment of the present invention, the second opening of the dressing is configured to form a seal around the conduit of the catheter assembly.

In accordance with an embodiment of the present invention, the perimeter of the dressing defines a shape that is not a square, and wherein the portions of the base defining the first opening are arranged such that the portions form an oblique angle.

In accordance with an embodiment of the present invention, the base and/or the cover portion of the dressing includes an adhesive on a bottom surface thereof.

In accordance with an embodiment of the present invention, the base and/or the cover portion of the dressing further includes a removable backing at least partially covering the adhesive.

In accordance with an embodiment of the present invention, the removable backing includes at least two sections, and wherein the first opening and/or the second opening at least partially separate the at least two sections.

In accordance with an embodiment of the present invention, the base of the dressing includes a non-woven material.

In accordance with an embodiment of the present invention, the base of the dressing defines an interior portion substantially having a shape of the catheter adapter.

In accordance with an embodiment of the present invention, the cover portion of the dressing further includes one or more alignment markers.

In accordance with an embodiment of the present invention, the base includes two alignment markers arranged on opposite sides of the interior.

In accordance with an embodiment of the present invention, a system includes a catheter assembly including a catheter adapter. The catheter adapter includes a distal end, a proximal end, a lumen arranged between and in fluid communication with the distal end and the proximal end, a port arranged between the proximal end and the distal end at an angle relative to the lumen, a conduit in fluid communication with the port, and a catheter arranged at the distal end of the catheter adapter and in fluid communication with the lumen. The system also includes a dressing for use with the catheter assembly, including a base defining a perimeter and an interior, the base including a bottom surface configured to contact skin of a patient and including a first opening in the perimeter, and a cover portion attached to the base and configured to cover a site of a catheter insertion, the cover portion including a second opening, the second opening configured to align with the first opening in the base when the dressing is applied to the patient’s skin.

In accordance with an embodiment of the present invention, the second opening of the dressing is configured to form a seal around a portion of a catheter assembly that passes through the first opening.

In accordance with an embodiment of the present invention, the second opening of the dressing is configured to form a seal around the conduit of the catheter assembly.

In accordance with an embodiment of the present invention, the perimeter of the dressing defines a shape that is not a square, and wherein the portions of the base defining the first opening are arranged such that the portions form an oblique angle.

In accordance with an embodiment of the present invention, the base and/or the cover portion of the dressing includes an adhesive on a bottom surface thereof.

In accordance with an embodiment of the present invention, the base and/or the cover portion of the dressing further includes a removable backing at least partially covering the adhesive.

In accordance with an embodiment of the present invention, the removable backing includes at least two sections, and wherein the first opening and/or the second opening at least partially separate the at least two sections.

In accordance with an embodiment of the present invention, the base of the dressing includes a non-woven material.

In accordance with an embodiment of the present invention, the base of the dressing defines an interior portion substantially having a shape of the catheter adapter.

In accordance with an embodiment of the present invention, the base and/or the cover portion of the dressing further includes one or more alignment markers.

In accordance with an embodiment of the present invention, the base comprises two alignment markers arranged on opposite sides of the interior.

### BRIEF DESCRIPTION OF THE DRAWINGS

FIG. 1 is a perspective view of a non-limiting embodiment of a catheter assembly with which non-limiting embodiments of dressings disclosed herein may be used;

FIG. 2 is a top view of a dressing according to non-limiting embodiments described herein;

FIG. 3 is a top view of a dressing according to non-limiting embodiments described herein;

FIG. 4 is a top view of a dressing and catheter assembly according to non-limiting embodiments described herein;

FIG. 5 is a top view of a dressing and catheter assembly according to non-limiting embodiments described herein;

FIGS. 6A-6C are top views of a dressing according to non-limiting embodiments described herein;

FIGS. 7A-7D are top views of a dressing according to non-limiting embodiments described herein; and

FIG. 8 is a top view of a dressing and catheter assembly according to non-limiting embodiments described herein.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

It should be understood that any numerical range recited herein is intended to include all values and sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10.

Provided herein are dressings, and kits and systems including such dressings, adapted for use with catheter assemblies, in particular catheter assemblies including extension sets. Dressings as described herein may be used to provide a secure site of attachment of a catheter adapter to a site of a venipuncture, and may provide improved visualization of catheter adapters, extension sets, and medical interventions that are delivered through such extension sets and/or catheter adapters.

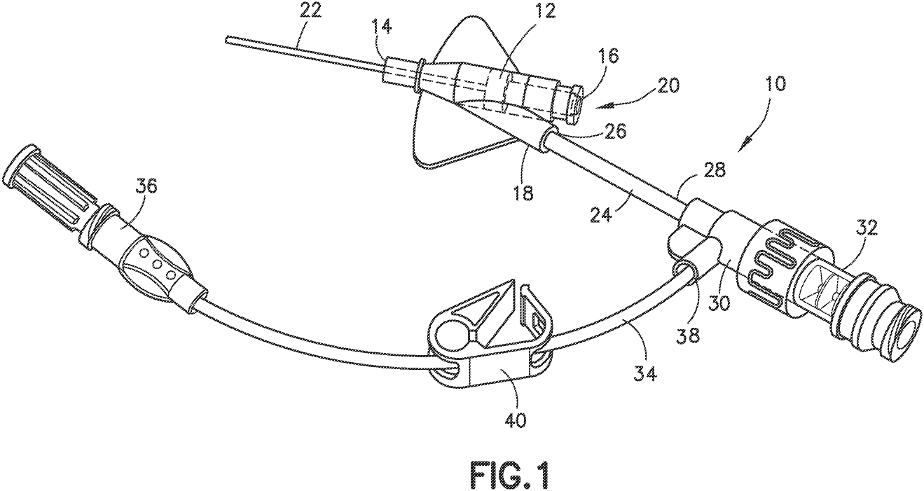

Referring now to FIG. 1, shown is a non-limiting embodiment of a catheter assembly 10 useful with stabilization devices as described herein below. Cather assembly 10 may include a catheter adapter 12, which may include a distal end 14 and a proximal end 16. In some embodiments, the catheter adapter 12 may include an additional port 18. In some embodiments, port 18 may disposed between the distal end 14 and the proximal end 16. In some embodiments, port 18 may be disposed at proximal end 16. In some embodiments, the first catheter adapter 12 may include a first lumen 20 extending through the distal end 14 and the proximal end 16.

In some non-limiting embodiments or aspects, the catheter assembly 10 may include a catheter 22 extending from the distal end 14. In some embodiments, the first catheter 22 may include a peripheral intravenous catheter, a midline catheter, or a peripherally-inserted central catheter. Catheter 22 may be formed of any suitable material and may be of any useful length, as known to those of skill in the art. In some non-limiting embodiments or aspects, the catheter assembly 10 may include a first fluid conduit 24 extending from the port 18. First fluid conduit 24 may be formed of any suitable material known to those of skill in the art, and may have a distal end 26 and a proximal end 28, and first fluid conduit 24 may be coupled, at distal end 26 thereof, to port 18. In some non-limiting embodiments or aspects, a connector 30 may be coupled to a proximal end 28 of first fluid conduit 24. Connector 30 may be a t-connector (e.g., one side port arranged at a 90 degree angle relative to a longitudinal axis of connector 30), a y-connector (e.g., one side port arranged at a 25, a 60, or a 75 degree angle relative to a longitudinal axis of connector 30), or any other type of connector known in the art, and may include a second lumen therethrough, having any number of branches suitable for the type of connector.

In some non-limiting embodiments or aspects, catheter assembly 10 may include an extension set including a second fluid conduit 34. In non-limiting embodiments, connector 30 includes a port 38 to which an extension set (e.g., second fluid conduit 34) may be connected. Extension sets 34 are known to those of skill in the art and are commercially available from, for example, Becton, Dickinson and Company under the tradenames MAXPLUS, MAXZERO, NEUTRACLEAR, Q-SYTE, and SMARTSITE. In some non-limiting embodiments or aspects, second fluid conduit 34 may include a luer connection 36 at an end thereof. In some non-limiting embodiments or aspects, the extension set may include a clamp 40, to allow for occlusion of second fluid conduit 34. Clamp 40 and second fluid conduit 34 may be formed of any suitable materials known to those of skill in the art. In non-limiting embodiments, second lumen (e.g., within connector 30) has an inner diameter that is substantially equivalent to an inner diameter of first fluid conduit 24 and/or second fluid conduit 34. Catheter assembly 10 may include a needleless access connector 32 and/or a second fluid conduit 34. Needleless access connectors 32 are known to those of skill in the art and are commercially available from, for example, Becton, Dickinson and Company under the tradenames MAXPLUS, MAXZERO, Q-SYTE, and SMARTSITE.

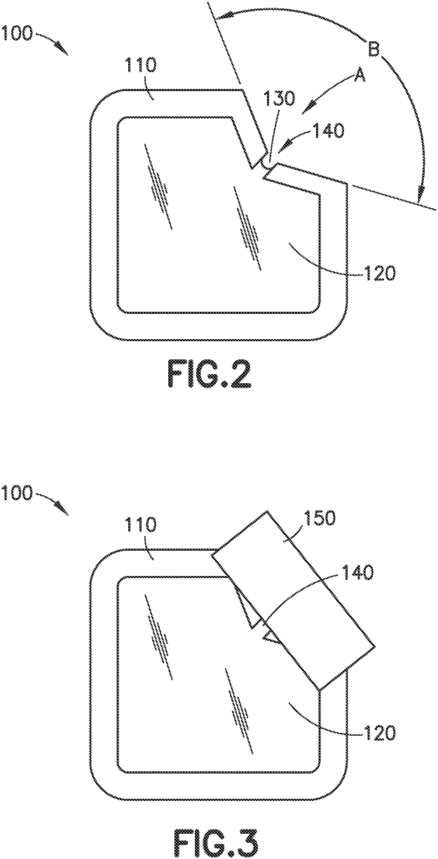

Turning to FIG. 2, shown is a non-limiting embodiment of an optimized dressing 100, including a base 110 and a cover portion 120. Base 110 may be formed of any suitable material known to those of skill in the art, including fabrics, optionally non-woven materials. Base 110 may define a perimeter of any suitable shape. In non-limiting embodiments, base 110 defines a shape that is not a parallelogram. In non-limiting embodiments, for example as shown in FIG. 2-6C, base 110 defines a perimeter having a shape that includes, along at least one side thereof, a cut-out region A defining an angle B that is optionally obtuse. In non-limiting embodiments, base 110 includes a top surface and a bottom surface, the bottom surface configured to contact skin of a patient onto which integrated dressing 100 is applied. In non-limiting embodiments, base 110 includes one or more suitable adhesives known to those of skill in the art on at least a portion of the bottom surface.

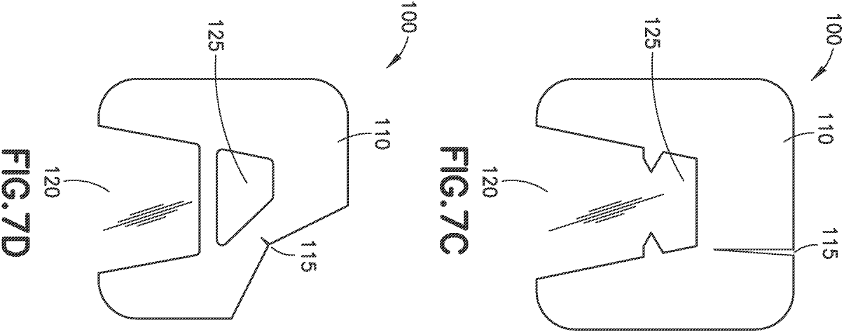

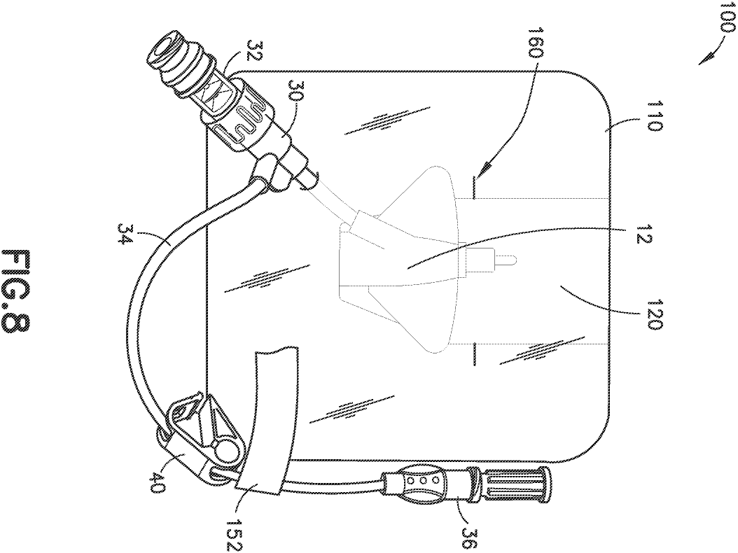

In non-limiting embodiments, base 110 includes a first opening 130, optionally only a single opening, configured to allow at least a portion of a catheter assembly, such as catheter assembly 10, optionally first fluid conduit 24, to pass therethrough. In non-limiting embodiments, first opening 130 is arranged in an area defined by angle B in the perimeter of base 110, optionally at the vertex of the obtuse angle, for example as shown in the accompanying figures. In non-limiting embodiments, for example as shown in FIG. 7A-8, base 110 may define an interior alignment window 125, which may be substantially shaped like a catheter adapter, such as catheter adapter 12 shown in FIG. 1.

In some non-limiting embodiments, portions of base 110 that include an adhesive are covered by removable tabs (e.g., a backing). In non-limiting embodiments, the backing comprises two portions, optionally separate and distinct portions, where an opening, e.g., first opening 130 defines a separation between the backings. In non-limiting embodiments, the one or more adhesives may include one or more antimicrobial compositions. Suitable antimicrobial compositions are known to those of skill in the art and may include, for example, chlorhexidine gluconate (CHG).

With continuing reference to FIG. 2, cover portion 120 may be coupled to base 110 at one or more locations along the perimeter defined by base 110, or substantially continuously along the perimeter. In some non-limiting embodiments, cover portion 120 is substantially or completely transparent, to allow for visualization of catheter assembly 10 arranged thereunder. In non-limiting embodiments, cover portion 120 includes an adhesive, optionally the same adhesive as provided on base 110, on a bottom surface thereof, to allow cover portion 120 to be applied to and to adhere to base 110, the patient’s skin, and/or one or more components of catheter assembly 10. In non-limiting embodiments, cover portion 120 is provided with base 110 as a single component, prepackaged as a single dressing. In non-limiting embodiments, cover portion 120 includes a second opening 140 through which at least a portion of a catheter assembly, such as catheter assembly 10, optionally first fluid conduit 24, may pass therethrough. Second opening 140 may be configured such that it can form a seal around the portion of catheter assembly 10 passing therethrough, thereby lessening the opportunity for infection at the site of catheter insertion.

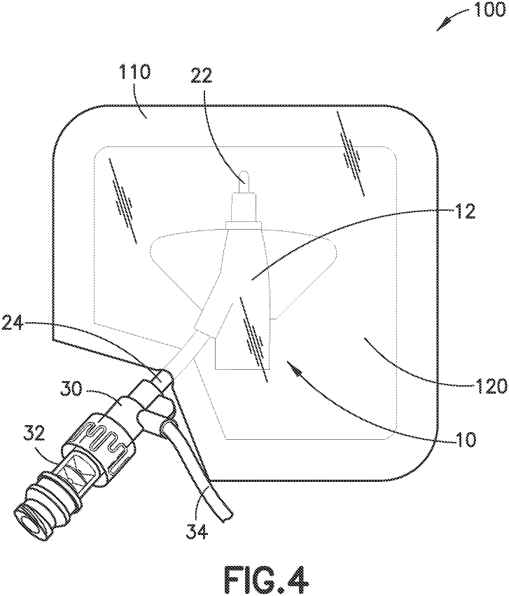

Turning to FIG. 3, in non-limiting embodiments, dressing 100 may include as part of packaging, or as part of a kit with a catheter assembly 10, a secondary dressing or tape 150. Secondary dressing or tape 150 may be a fabric, optionally a non-woven material, and may be the same material as base 110. Secondary dressing or tape 150 may be applied underneath the component of catheter assembly 10 that passes through first opening 130. The inclusion of secondary dressing or tape 150, in combination with opening 130, provides an improved seal around portions of catheter assembly 10 that pass through opening 130, lessening the opportunity for infection. In addition, the inclusion of secondary dressing or tape 150 provides mechanical stability, limiting movement of catheter adapter 12 in relation to the patient’s skin, which reduces phlebitis and catheter dislodgment.

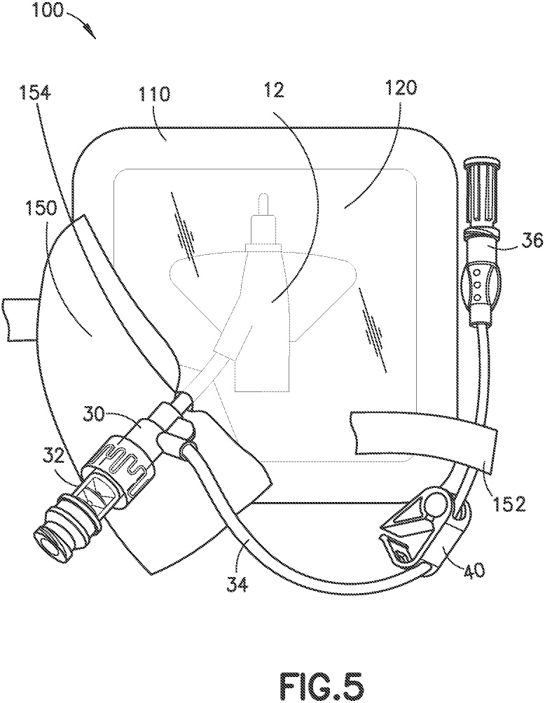

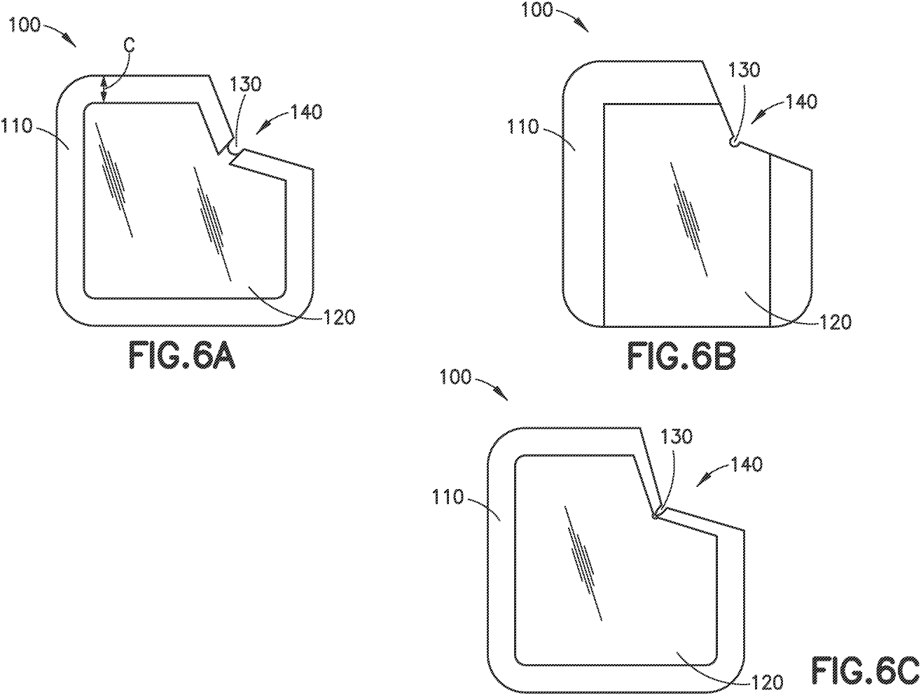

With regard to FIGS. 4 and 5, shown are embodiments of dressing 100 in use, applied covering a catheter assembly 10. As can be appreciated, arrangement of base 110 and cover portion 120 of dressing 100 allows for excellent visualization of catheter adapter 12, the patient’s skin around the catheter insertion site, and first fluid conduit 24. As noted previously, inclusion of secondary dressing or tape 150, in combination with opening 130, provides an improved seal 154 around portions of catheter assembly 10 that pass through opening 130, lessening the opportunity for infection. As can be seen in the non-limiting embodiment of FIG. 5 (as well as FIG. 8), a tertiary dressing or tape 152 may be included to secure second fluid conduit 34.

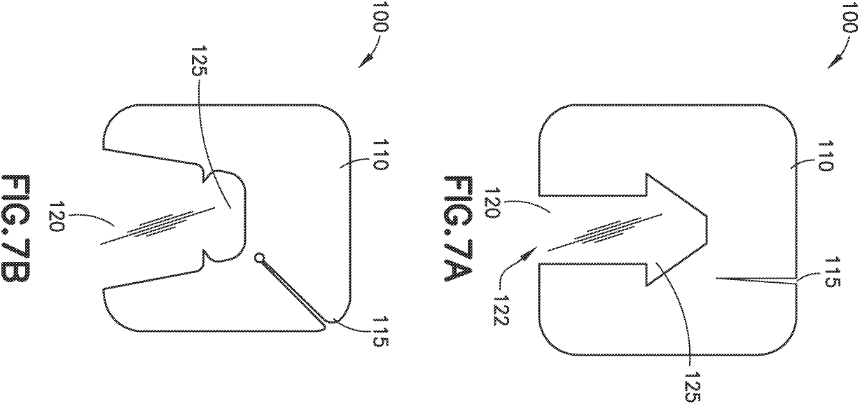

Turning to FIGS. 6A-6C, shown are other non-limiting embodiments of dressing 100. Specifically, the embodiments shown in FIGS. 6A-6C include different arrangements of base 110 at and around first opening 130. In FIG. 6A, base 110 has a uniform thickness C substantially around the entirety of the perimeter thereof, including in cut-out region present in other embodiments, such as those denoted A in FIG. 2, at and around first opening 130. In FIG. 6B, a first opening 130 is significantly larger than in FIG. 6A, as base 110 is not present in cut-out region A. In FIG. 6C, base 110 has a greater thickness C along perimeter thereof in regions outside of cutout region A.

With regard to FIG. 7A-8, shown are embodiments of dressing 100 in which base 110 defines an interior that differs from the open interior of the embodiments of, for example FIGS. 6A-6C. In the embodiments of FIG. 7A-8, interior of base 110 defines an opening 122, which may include an alignment window 125. Such a window 125 may aid a medical professional in correctly aligning dressing 100 about catheter adapter 12. In non-limiting embodiments,opening 122 is substantially u-shaped. In non-limiting embodiments, alignment window 125 includes at least a portion substantially shaped like a catheter adapter, such as catheter adapter 12 shown in FIG. 1. As shown in FIGS. 7A-7D, opening 122 and alignment window 125 may assume a number of different configurations, including where portions of base 110 protrude inwardly into opening 122 to at least partially define alignment window 125 (FIGS. 7B-7C), and where alignment window 125 is completely separated from opening 122 by a portion of base 110 (FIG. 7D). In the illustrated embodiments of FIG. 7A-8, first opening 130 may be replaced by a slit 115, which may serve the same function (e.g., allowing a component of a catheter assembly to pass therethrough, for improved visualization).

In non-limiting embodiments of any of the aforementioned dressings, alignment marker(s) 160 may be arranged on base 110 and/or cover portion 120 of dressing 100. In non-limiting embodiments, such alignment markers function to provide an indication to a medical professional of any displacement of dressing 100 and/or catheter adapter 12, relative to the site of insertion of catheter 22. This can aid a medical professional in determining whether dressing 100 and/or catheter adapter 12 has moved an unacceptable difference (e.g., raising issues of sufficiently preventing opportunity for infection or of decreased use (e.g., kinking, dislodgment) of catheter 22 that can limit the ability to introduce medical devices (e.g., devices for use in line draw applications) and/or medicaments into the patient through catheter 22 and/or limit the ability to withdraw blood through catheter 22), such that dressing 100 and/or catheter should be removed and/or reapplied.

Although the present disclosure has been described in detail for the purpose of illustration based on what is currently considered to be the most practical and preferred embodiments or aspects, it is to be understood that such detail is solely for that purpose and that the present disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment may be combined with one or more features of any other embodiment.

The invention claimed is:

1. A dressing for use with a catheter assembly, comprising:
a base defining a perimeter and an interior, the base comprising a bottom surface configured to contact skin of a patient and comprising a first opening in the perimeter; and
a cover portion attached to the base and configured to cover a site of a catheter insertion, the cover portion comprising a second opening, the second opening configured to align with the first opening in the base when the dressing is applied to the patient’s skin.

2. The dressing of claim 1, wherein the second opening is configured to form a seal around a portion of a catheter assembly that passes through the first opening.

3. The dressing of claim 1, wherein the perimeter defines a shape that is not a square, and wherein the portions of the base defining the first opening are arranged such that the portions form an oblique angle.

4. The dressing of claim 1, wherein the base and/or the cover portion comprise an adhesive on a bottom surface thereof.

5. The dressing of claim 4, wherein the base and/or the cover portion further comprise a removable backing at least partially covering the adhesive.

6. The dressing of claim 5, wherein the removable backing comprises at least two sections, and wherein the first opening and/or the second opening at least partially separate the at least two sections.

7. The dressing of claim 1, wherein the base comprises a non-woven material.

8. The dressing of claim 1, wherein the base defines an interior portion substantially having a shape of a catheter adapter.

9. The dressing of claim 1, wherein the base and/or the cover portion further comprise one or more alignment markers.

10. The dressing of claim 9, wherein the base comprises two alignment markers arranged on opposite sides of the interior.

11. A kit, comprising:
at least one package containing:
a catheter assembly comprising:
a catheter adapter, comprising:
a distal end;
a proximal end;
a lumen arranged between and in fluid communication with the distal end and the proximal end;
a port arranged between the proximal end and the distal end
at an angle relative to the lumen;
a conduit in fluid communication with the port; and
a catheter arranged at the distal end of the catheter adapter and in fluid communication with the lumen; and
a dressing having a base defining a perimeter and an interior, the base comprising a bottom surface configured to contact skin of a patient and comprising a first opening in the perimeter, and
a cover portion attached to the base and configured to cover a site of a catheter insertion, the cover portion comprising a second opening, the second opening configured to align with the first opening in the base when the dressing is applied to the patient’s skin.

12. The kit of claim 11, wherein the second opening of the dressing is configured to form a seal around a portion of a catheter assembly that passes through the first opening.

13. The kit of claim 12, wherein the second opening of the dressing is configured to form a seal around the conduit of the catheter assembly.

14. The kit of claim 11, wherein the perimeter of the dressing defines a shape that is not a square, and wherein the portions of the base defining the first opening are arranged such that the portions form an oblique angle.

15. The kit of claim 11, wherein the base and/or the cover portion of the dressing comprise an adhesive on a bottom surface thereof.

16. The kit of claim 15, wherein the base and/or the cover portion of the dressing further comprise a removable backing at least partially covering the adhesive.

17. The kit of claim 16, wherein the removable backing comprises at least two sections, and wherein the first opening and/or the second opening at least partially separate the at least two sections.

18. The kit of claim 11, wherein the base of the dressing comprises a non-woven material.

19. The kit of claim 11, wherein the base of the dressing defines an interior portion substantially having a shape of the catheter adapter.

20. The kit of claim 11, wherein the base and/or the cover portion of the dressing further comprise one or more alignment markers.
